(19) United States
(12) Patent Application Publication
(10) Pub. No.: US 2023/0408616 A1
(43) Pub. Date: Dec. 21, 2023

(54) SINGLE-VOXEL SPECTROSCOPY FOR QUANTITATION OF MYOCARDIAL METABOLITES

(71) Applicants: Siemens Healthcare GmbH, Erlangen (DE); Beth Israel Deaconess Medical Center, Inc., Boston, MA (US)

(72) Inventor: Xiaoying Cai, Brookline, MA (US)

(21) Appl. No.: 18/049,720

(22) Filed: Oct. 26, 2022

Related U.S. Application Data
(60) Provisional application No. 63/366,553, filed on Jun. 17, 2022.

(51) Int. Cl. G01R 33/567; G01R 33/385; A61B 5/055

(52) U.S. Cl. CPC G01R 33/5673; G01R 33/385; A61B 5/055

(57) ABSTRACT

A system for dark-blood single-voxel magnetic resonance (MR) spectroscopy includes application of double inversion recovery preparation pulses to the subject during a first R-R interval from a first R peak to a second R peak, application of a plurality of localization pulses to a portion of the subject during a second R-R interval from the second R peak to a third R peak, wherein application of the plurality of localization pulses is triggered by elapsing of a first delay time from the second R peak, the first delay time determined to substantially null a blood signal emitted from the portion of the subject, acquisition of MR data from the portion of the subject, and determination of a quantity of one or more metabolites in the region of interest based on the acquired MR data.

### CROSS-REFERENCE TO RELATED APPLICATIONS

This application claims priority to U.S. Provisional Patent Application No. 63/366,553, filed Jun. 17, 2022, the disclosure of which is incorporated herein by reference for all purposes.

### BACKGROUND

Proton (¹H) magnetic resonance (MR) single-voxel spectroscopy has been used to detect and quantify metabolites in vivo. In one specific example, single-voxel spectroscopy is used to detect and quantitate creatine and myocardial triglycerides within a particular voxel of the cardiac septum. Localization of the voxel is performed by applying either point-resolved spectroscopy (PRESS) or stimulated echo acquisition mode (STEAM) pulse sequences. Typically, two scans are performed, where the first scan measures the metabolite peak area and the second scan measures the water peak area. The triglycerides content is determined as the ratio of the metabolite peak area to the water peak area.

Accurate quantitation of metabolites in heart tissue is particularly challenging due to respiratory, cardiac, body and other motion, which causes movement of the voxel of interest. The effects of respiratory motion may be reduced by using a navigator to monitor respiration states and to perform gating based thereon. Similarly, cardiac motion can be mitigated by using ECG triggering to synchronize the localization and signal readout to a consistent phase of the cardiac cycle. However, signal loss is unavoidable as the heart is constantly moving during the cardiac cycle. Moreover, the degree of signal loss may vary depending on the trigger delay, which introduces additional uncertainty in the quantitation process.

The voxel of interest is typically placed on the septum to avoid signals from the blood pool. However, such techniques may be unreliable because the motion of the blood in the voxel is unpredictable and dependent on various factors, such as trigger time and function of the myocardium. Moreover, if the spectroscopy also includes the use of known motion-compensating methods, these methods may increase the signal from blood and cause further bias in the quantitation, particularly if the voxel partially includes the blood pool due to motion or inaccurate localization.

Improved single-voxel spectroscopy for quantitation of myocardial metabolites is desired.

### BRIEF DESCRIPTION OF THE DRAWINGS

FIG. 1 is a block diagram of an example MRI system for use in some embodiments.

FIG. 2 illustrates a pulse sequence for dark-blood single-voxel spectroscopy with double inversion pulses in some embodiments.

FIG. 3 is a graph of trigger delay for double inversion recovery pulses versus heart rate in some embodiments.

FIG. 4 is a flow diagram of a process to execute a pulse sequence for dark-blood single-voxel spectroscopy with double inversion pulses in some embodiments.

FIG. 5 illustrates a pulse sequence for dark-blood single-voxel spectroscopy with double inversion pulses and navigator pulses in some embodiments.

FIG. 6 is a flow diagram of a process to execute a pulse sequence for dark-blood single-voxel spectroscopy with double inversion pulses and navigator pulses in some embodiments.

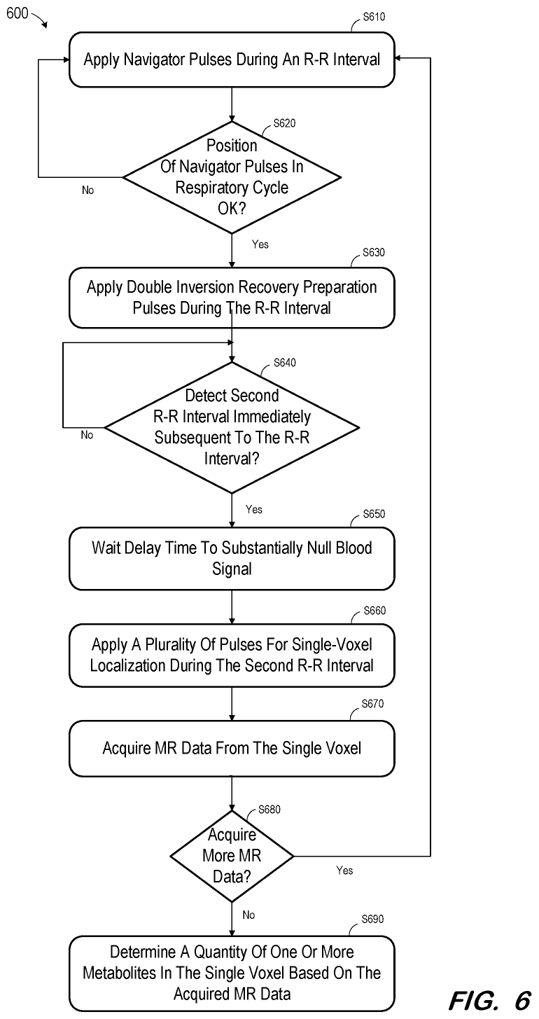

FIG. 7 illustrates a pulse sequence for dark-blood single-voxel spectroscopy with double inversion pulses and navigator pulses in some embodiments.

FIG. 8 is a flow diagram of a process to execute illustrates a pulse sequence for dark-blood single-voxel spectroscopy with double inversion pulses and navigator pulses in some embodiments.

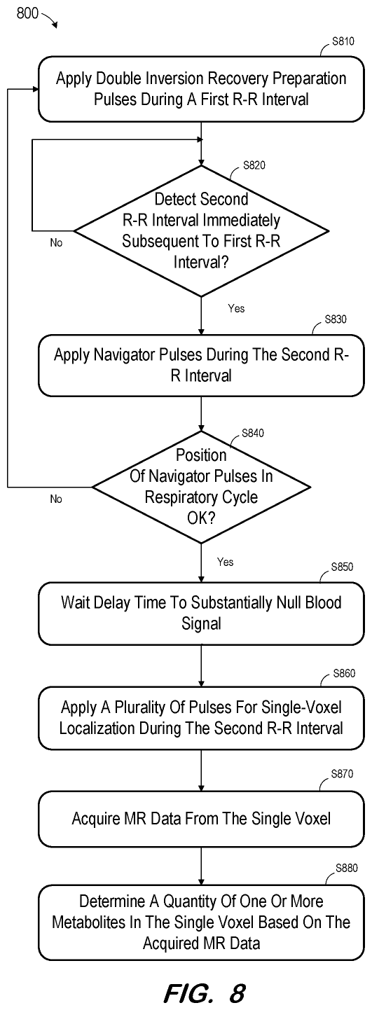

### DETAILED DESCRIPTION

The following description is provided to enable any person in the art to make and use the described embodiments. Various modifications will remain apparent to those in the art.

It would be desirable to provide a system and method for dark-blood and motion-compatible single-voxel MR spectroscopy suitable for quantitation of myocardial triglycerides or other metabolite content.

In some embodiments, single-voxel MR spectroscopy includes performing, using a magnetic resonance imaging (MRI) system, a dark-blood single-voxel spectroscopy pulse sequence to acquire MR data from a region of interest in a subject. The pulse sequence can include dual inversion preparation pulses applied in a first R-R interval followed by excitation pulses including a plurality of localization pulses for spectroscopy in a second successive R-R interval. The timing of the plurality of localization pulses may be configured to null blood signal, and a quantitation of metabolite content of the region of interest is generated based on the thusly-acquired MR data.

In some embodiments, single-voxel spectroscopy is used for imaging of other body parts where suppressing signal contribution from blood could also improve the accuracy of quantitation. Some embodiments may achieve accurate and reproducible quantitation of myocardial triglycerides content in combination with the second-order motion compensated localization. The plurality of localization pulses may comprise pulses conforming to point-resolved spectroscopy (PRESS) techniques, for example, for quantitating myocardial triglycerides. In some embodiments, the plurality of localization pulses may comprise pulses conforming to stimulated echo acquisition mode (STEAM) technique, for quantitating other metabolites, such as creatine. Some embodiments further utilize navigator pulses for respiratory motion control.

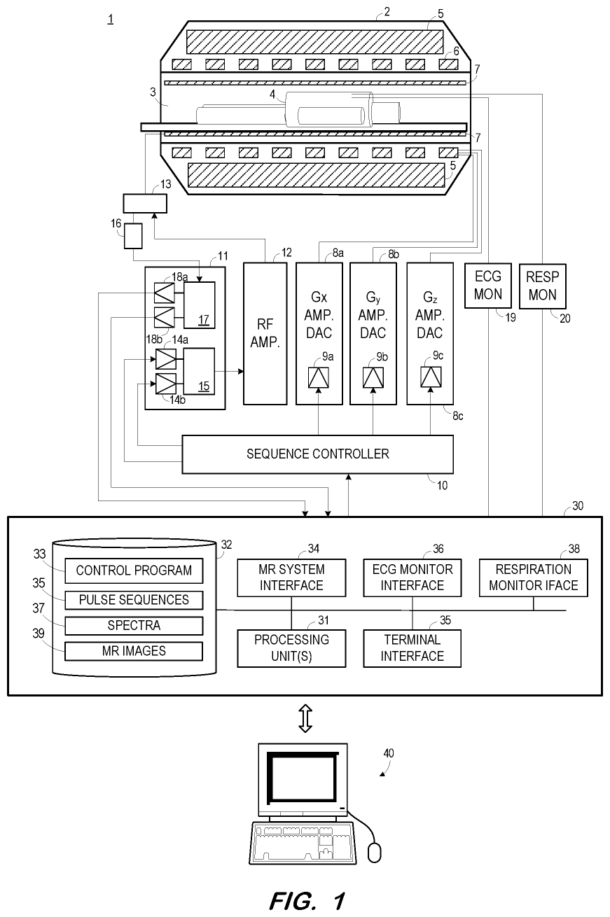

FIG. 1 illustrates MR system 1 according to some embodiments. MR system 1 includes MR chassis 2, which defines bore 3 in which patient 4 is disposed. MR chassis 2 includes polarizing main magnet 5, gradient coils 6 and RF coil 7 arranged about bore 3. According to some embodiments, polarizing main magnet 5 generates a uniform main magnetic field (B₀) and RF coil 7 emits an excitation field (B₁).

According to MR techniques, a substance (e.g., human tissue) is subjected to a main polarizing magnetic field (i.e., B₀), causing the individual magnetic moments of the nuclear spins in the substance to process about the polarizing field in random order at their characteristic Larmor frequency, in an attempt to align with the field. A net magnetic moment M_{z} is produced in the direction of the polarizing field, and the randomly-oriented magnetic components in the perpendicular plane (the x-y plane) cancel out one another.

The substance is then subjected to an excitation field (i.e., B₁) created by emission of a radiofrequency (RF) pulse, which is in the x-y plane and near the Larmor frequency, causing the net aligned magnetic moment M_{z} to rotate into the x-y plane so as to produce a net transverse magnetic moment Mₜ, which is rotating, or spinning, in the x-y plane at the Larmor frequency. The excitation field is terminated, and signals are emitted by the excited spins as they return to their pre-excitation field state. The emitted signals are detected, digitized and processed to reconstruct an image or a spectrum using one of many well-known MR techniques.

Gradient coils 6 produce magnetic field gradients Gₓ, G_{y}, and G_{z} which are used for position-encoding NMR signals. The magnetic field gradients Gₓ, G_{y}, and G_{z} distort the main magnetic field in a predictable way so that the Larmor frequency of nuclei within the main magnetic field varies as a function of position. Accordingly, an excitation field B₁ which is near a particular Larmor frequency will tip the net aligned moment M_{z} of those nuclei located at field positions which correspond to the particular Larmor frequency, and signals will be emitted only by those nuclei after the excitation field B₁ is terminated.

Gradient coils 6 may consist of three windings, for example, each of which is supplied with current by an amplifier 8a-8c in order to generate a linear gradient field in its respective Cartesian direction (i.e., x, y, or z). Each amplifier 8a-8c includes a digital-analog converter 9a-9c which is controlled by a sequence controller 10 to generate desired gradient pulses at prescribed times.

Sequence controller 10 also controls the generation of RF pulses by RF system 11 and RF power amplifier 12. RF system 11 and RF power amplifier 12 are responsive to a scan prescription and direction from sequence controller 10 to produce RF pulses of the desired frequency, phase, and pulse amplitude waveform. The generated RF pulses may be applied to the whole of RF coil 7 or to one or more local coils or coil arrays. RF coil 7 converts the RF pulses emitted by RF power amplifier 12, via multiplexer 13, into a magnetic alternating field to excite the nuclei and align the nuclear spins of the object to be examined or the region of the object to be examined. As mentioned above, RF pulses may be emitted in a magnetization preparation step to enhance or suppress certain signals.

The RF pulses are represented digitally as complex numbers. Sequence controller 10 supplies these numbers in real and imaginary parts to digital-analog converters 14a-14b in RF system 11 to create corresponding analog pulse sequences. Transmission channel 15 modulates the pulse sequences with a radio-frequency carrier signal having a base frequency corresponding to the resonance frequency of the nuclear spins in the volume to be imaged.

RF coil 7 both emits radio-frequency pulses as described above and scans the alternating field which is produced because of precessing nuclear spins, i.e., the nuclear spin echo signals. The received signals are received by multiplexer 13, amplified by RF amplifier 16 and demodulated in receiving channel 17 of RF system 11 in a phase-sensitive manner. Analog-digital converters 18a and 18b convert the demodulated signals into digitized real and imaginary components.

Electrocardiograph (“ECG”) monitor 19 acquires ECG signals from electrodes placed on patient 4 and respiratory monitor 20 acquires respiratory signals from a respiratory bellows or other respiratory monitoring device. Such physiological signals may be used by sequence controller 10 to synchronize, or “gate”, transmitted RF pulses of a spectroscopy pulse sequence based on the heartbeat and/or respiration of patient 4 as described herein.

Computing system 30 receives the digitized real and imaginary components from analog-digital converters 18a and 18b and may process the components according to known techniques. Such processing may, for example, include reconstructing two-dimensional or three-dimensional images by performing a Fourier transformation of raw k-space data, performing other image reconstruction techniques such as iterative or back-projection reconstruction techniques, applying filters to raw k-space data or to reconstructed images, generating functional magnetic resonance images, calculating motion or flow images, and generating a chemical shift vs. magnitude spectrum.

System 30 may comprise any general-purpose or dedicated computing system. Accordingly, system 30 includes one or more processing units 31 (e.g., processors, processor cores, execution threads, etc.) configured to execute processor-executable program code to cause system 30 to operate as described herein, and storage device 32 for storing the program code. Storage device 32 may comprise one or more fixed disks, solid-state random access memory, and/or removable media (e.g., a thumb drive) mounted in a corresponding interface (e.g., a USB port).

One or more processing units 31 may execute program code of control program 33 to provide instructions to sequence controller 10 via MR system interface 34. For example, sequence controller 10 may be instructed to initiate a desired pulse sequence of pulse sequences 35. In particular, sequence controller 10 may be instructed to control the switching of magnetic field gradients via amplifiers 8a-8c at appropriate times, the transmission of radio-frequency pulses having a specified phase and amplitude at specified times via RF system 11 and RF amplifier 12, and the readout of the resulting MR signals. The timing of the various pulses of a pulse sequence may be based on physiological data received by ECG monitor interface 36 and/or respiratory monitor 38.

Storage device 32 stores spectra 37 generated as described herein and MR images 39. Such spectra and images may be provided to terminal 40 via terminal interface 35 of system 30. Terminal interface 35 may also receive input from terminal 40, which may be used to provide commands to control program 33 to initiate single-voxel spectroscopy as described herein. Terminal 40 may comprise a display device and an input device coupled to system 30. In some embodiments, terminal 40 is a separate computing device such as, but not limited to, a desktop computer, a laptop computer, a tablet computer, and a smartphone.

Each element of system 1 may include other elements which are necessary for the operation thereof, as well as additional elements for providing functions other than those described herein. Storage device 22 may also store data and other program code for providing additional functionality and/or which are necessary for operation of system 20, such as device drivers, operating system files, etc.

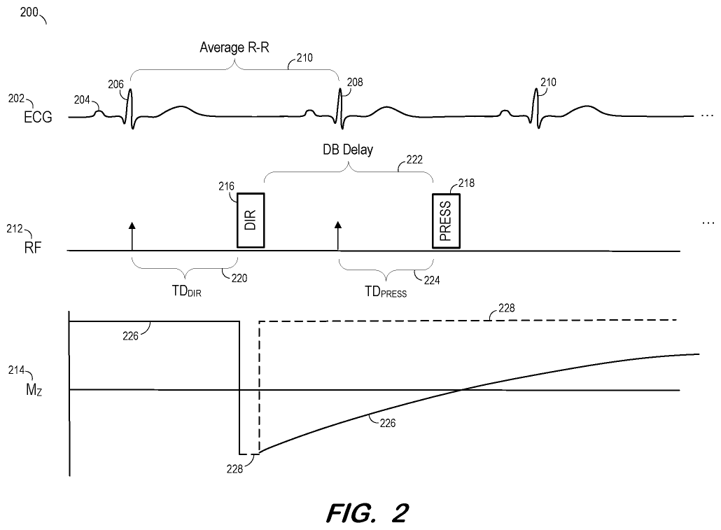

FIG. 2 illustrates a pulse sequence for dark-blood single-voxel spectroscopy with double inversion recovery preparation pulses in accordance with some embodiments. The pulse sequence 200 can be performed, for example, using an MRI system such as but not limited to system 1 to acquire MR data (or signals) from a subject. While the following description is directed to an application for the myocardium, it should be understood that the sequence 200 may be used to acquire MR data from other body parts

In FIG. 2, ECG axis 202 shows ECG signal 204 including R peaks 206, 208 and 210 of a subject. FIG. 2 also includes RF axis 212 and magnetization signal (M t) axis 214. Pulse sequence 200 includes double inversion recovery (DIR) preparation pulses 216 that are applied during an R-R interval between R peak 206 and R peak 208 to null the blood signal 226 and provide a dark blood effect. Excitation pulses 218 such as, for example, PRESS pulses are applied during an R-R interval between R peaks 208 and 210. Pulses 218 are applied after a delay time (DB delay 222) from application of pulses 216. Determination of delay time 222 will be described below. In some embodiments, pulses 218 conform to the STEAM technique.

In some embodiments, PRESS pulses 218 include three slice-selective RF pulses for localization. DIR pulses 216 may consist of a non-selective 180-degree RF pulse to invert all the magnetization and a selective 180-degree RF pulse applied immediately following the non-selective 180-degree RF pulse to restore the magnetization of the selected slice on the myocardium. The blood signal outside of the slice profile of the selective 180-degree RF pulse would then experience T₁-relaxation and is nulled with the calculated timings when it arrives in the ventricles during the second heartbeat 208. In some embodiments, the RF pulses 216 are configured so that the selected slice includes the cardiac septum.

Application of pulses 216 is triggered by elapsing of time delay TD_{DIR} 220 from R peak 206, and application of pulses 218 is triggered by elapsing of time delay TD_{PRESS} 224 from R peak 208. TD_{DIR} 220 and TD_{PRESS} 224 are determined such that the magnetization M_{z} of septal myocardium (represented by curve 228) is substantially undisturbed while the magnetization M_{z} of blood pool (represented by curve 226) is substantially nulled (e.g., is substantially close to the zero magnitude line of magnetization M_{z} axis 214) with T₁-relaxation at the end of DB delay 222 (i.e., before and/or during execution of pulses 218). TD_{DIR} 220 and TD_{PRESS} 224 may also or alternatively be determined such that the heart is in diastole during pulses 218. In some embodiments, TD_{DIR} 220 and TD_{PRESS} 224 may be determined based on the T₁ of the blood and the heart rate.

In some embodiments, the determination assumes that pulses 216 and pulses 218 are applied during every other heartbeat and the signal of blood reaches a steady state of:

$${M}_{SS} = \frac{1 - {e}^{- 2 R R / T 1}}{1 + {e}^{- 2 R R / T 1}} \qquad Eq . 1$$

where RR indicates the average R-to-R interval 210 and is dependent on the heart rate of the subject. Therefore, to null the blood signal, DB delay 222 between DIR pulses 216 and PRESS pulses 218 is given by:

$$DB delay = [ ln ( 2 ) - ln ( 1 + {e}^{- \frac{2 R R}{T 1}} ) ] * {T}_{1} - T {D}_{P R E S S} \qquad Eq . 2$$

where T₁ is the longitudinal relaxation time of blood and TD_{PRESS} 224 is the trigger delay between the R peak of heartbeat 208 and commencement of PRESS pulses 218. TD_{PRESS} may be defined as 250 ms to place PRESS pulses 218 at end-systole but embodiments are not limited thereto. Therefore, trigger delay TD_{DIR} 220 before DIR pulses 216 is given by:

$${TD}_{DIR} = RR - \{ [ ln ( 2 ) - ln ( 1 + {e}^{- \frac{2 R R}{T 1}} ) \} * {T}_{1} - {TD}_{PRESS} \} \qquad Eq . 3$$

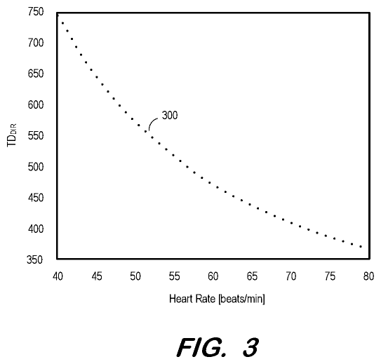

According, TD_{DIR} 220 is dependent on measured heart rate. FIG. 3 shows curve 300 of heart rate versus TD_{DIR} in some embodiments in which TD_{PRESS} is set to 250 ms. As shown, and evident from the above equations, TD_{DIR} and heart rate are inversely related.

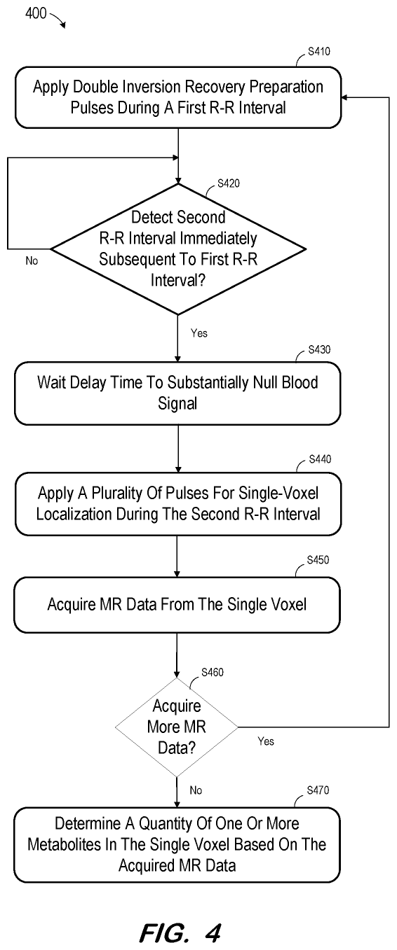

FIG. 4 comprises a flow diagram of process 400 to perform single-voxel spectroscopy according to some embodiments. In some embodiments, various hardware elements of system 1 (e.g., one or more processing units) execute program code to perform process 400. The steps of process 400 need not be performed by a single device or system.

Process 400 and all other processes mentioned herein may be embodied in executable program code read from one or more of non-transitory computer-readable media, such as a disk-based or solid-state hard drive, a DVD-ROM, a Flash drive, and a magnetic tape, and then stored in a compressed, uncompiled and/or encrypted format. In some embodiments, hard-wired circuitry may be used in place of, or in combination with, program code for implementation of processes according to some embodiments. Embodiments are therefore not limited to any specific combination of hardware and software.

An R-R interval of a subject is determined prior to process 400. The R-R interval may be determined by monitoring an ECG signal of the subject for a period of time after the subject is positioned in an MRI device. A value of T₁ (i.e., the longitudinal relaxation time of blood) and TD_{PRESS} (i.e., the delay between the detected R peak of a heartbeat and the commencement of single-voxel localization pulses are also determined prior to 400. The value of TD_{PRESS} may be defined based on the R-R interval so that the single-voxel localization pulses occur during a desired phase of the cardiac cycle (e.g., end-systole), but embodiments are not limited thereto. The R-R interval, the T₁ value and the TD_{PRESS} value may be used as described above to determine a time delay between the R peak of a heartbeat and commencement of dual inversion recovery pulses (i.e., TD_{DIR}).

At S410, double inversion recovery preparation pulses are applied to the subject during a first R-R interval. In one example of S410, an R peak of a heartbeat is detected and, after a period equal to TD_{DIR} elapses, the double inversion recovery preparation pulses are applied as described above and/or as known in the art. Flow then pauses at S420 to detect a next R-R interval, for example by detecting a next R peak.

Once the next R peak is detected, process 400 waits at S430 for a delay time required to substantially null the signal from blood in the voxel of interest. This delay time is TD_{PRESS} as described above. A plurality of pulses for single-voxel localization are applied during the next R-R interval and after the expiration of TD_{PRESS} at S440. The plurality of pulses may comprise a PRESS module, a STEAM module, or any other suitable voxel-localization modules. MR data is acquired from the voxel as a result of the applied pulses at S450. The MR data includes digitized real and imaginary components as is known in the art.

Next, at S460, it is determined whether more data is to be acquired. In this regard, the MR data acquired at S450 typically consists of a subset of k-space lines. Accordingly, if all desired k-space lines have not yet been acquired, flow returns to S410 and continues as described above to acquire additional k-space lines at S450. Flow proceeds from S460 to S470 once all desired k-space lines have been acquired.

At S470, a quantity of one or more metabolites in the single voxel is determined based on the acquired MR data. S470 may comprise generating a spectrum of chemical shift versus magnitude based on the MR data and determining the one or more quantities as is known in the art. The determined one or more quantities may be presented to a user via a terminal such as terminal 40.

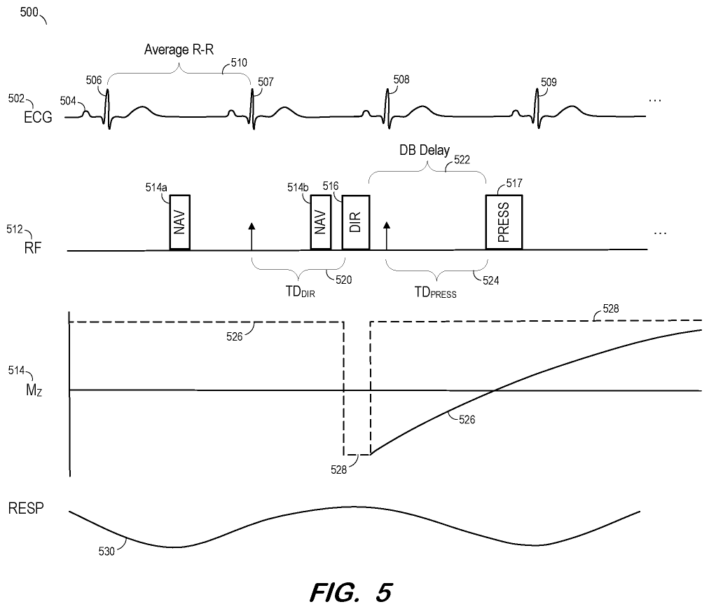

Some embodiments combine the above-described pulses with navigator pulses to enable free-breathing MR data acquisition. FIG. 5 illustrates pulse sequence 500 in which the navigator pulses are applied before the double inversion recovery pulses within an R-R interval. Any navigator pulses that are or become known may be used in some embodiments.

More particularly, FIG. 5 shows ECG axis 502 including a plurality of R peaks 506, 507, 508 and 509, RF axis 512, M_{z} axis 514, and respiratory motion waveform 530. Navigator pulses 514 are applied in a first R-R interval between peaks 506 and 507. Next, it is determined based on respiration waveform 530 whether the temporal position of pulses 514a coincides with the phase of respiratory motion (e.g., inspiration or expiration) at which data acquisition is desired. It will be assumed in this example the temporal position of pulses 514a is not suitable, therefore no DIR pulses are applied during the same R-R interval as pulses 514a and voxel-localization pulses are not applied in an immediately-subsequent R-R interval.

Second navigator pulses 514b are applied in a next R-R interval, at a temporal position corresponding to a different phase of respiratory waveform 530. It will be assumed that this temporal position is determined to be suitable, and therefore DIR pulses 516 are applied once TD_{DIR} 520 elapses after the occurrence of R peak 507 as shown in FIG. 5. Accordingly, application of navigator pulses in this embodiment should conclude in time to provide enough time for application of the DIR pulses between the elapsing of TD_{DIR} and a next R peak.

PRESS pulses 517 are then applied in a next interval, after R peak 508 and elapsing of TD_{PRESS} 524. In some embodiments, the application of the navigator pulses, double inversion recovery pulses and voxel-localization pulses may continue in this manner until all necessary MR data has been acquired.

Process 600 may be used to execute a sequence such as sequence 500 in some embodiments. As described above, values of TD_{DIR} and TD_{PRESS} are determined prior to process 600.

Navigator pulses are applied during an R-R interval at S610 and a navigator image is generated therefrom. The navigator image is reviewed at S620 to determine whether the navigator pulses were applied at the desired temporal position of respiration cycle (e.g., when the lung position corresponds to end-expiration). If not, flow returns to S610 where navigator pulses are applied during a next R-R interval and preferably during a different phase of the respiratory cycle.

If the temporal position of the navigator pulses is suitable, double inversion recovery preparation pulses are applied at S630. The double inversion recovery preparation pulses are applied during a same R-R interval as the prior-applied navigator pulses, and after the elapsing of TD_{DIR} from the first R peak of that R-R interval.

S640 through S690 may proceed as described above with respect to S420 through S470. However, if it is determined at S680 that all desired k-space lines have not yet been acquired, flow returns to S610 to apply navigator pulses during a subsequent R-R interval and continues to acquire another set of k-space lines.

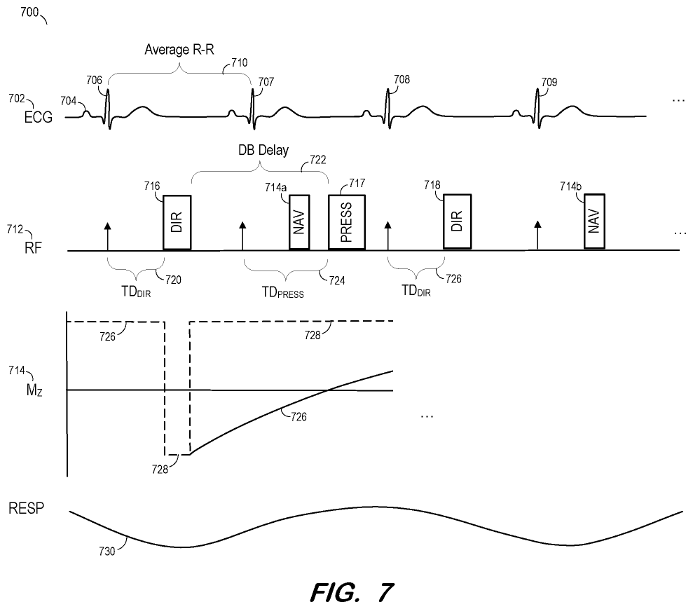

FIG. 7 illustrates the combination of navigator pulses with double inversion recovery pulses and voxel-location pulses for MR spectroscopy according to some embodiments.

ECG axis 702 includes R peaks 706, 707, 708 and 709, RF axis 712, M_{z} axis 714, and respiratory motion waveform 730. In response to detection of R peak 706, double inversion recovery pulses 716 are applied after elapsing of TD_{DIR} 720 as shown.

Navigator pulses 714a are then applied after detection of next R peak 707. As described above, it is determined whether the temporal position of pulses 714a is suitable in view of the phase of respiratory motion waveform 730 at this same temporal position. It will be assumed that the temporal position of pulses 714a is determined to be suitable, therefore PRESS pulses 717 are applied after elapsing of TD_{PRESS} 724 from R peak 707. Application of navigator pulses in this embodiment should conclude in time to provide enough time for application of the PRESS pulses between the elapsing of TD_{PRESS} and a next R peak.

Double inversion recovery pulses 718 applied in a next R-R interval, again after elapsing of TD_{DIR} 726 from the first R peak 708 of the R-R interval. Navigator pulses 714b are then applied after detection of next R peak 709 at a temporal position which may correspond to a different phase of respiratory waveform 730. It will be assumed that this temporal position is determined to be unsuitable, and therefore PRESS pulses are not applied in the same R-R interval as pulses 714b. Accordingly, no MR data is acquired in this R-R interval. In some embodiments, the application of the navigator pulses, double inversion recovery pulses and voxel-localization pulses may continue in this manner until all necessary MR data has been acquired.

Process 800 may be used to execute a sequence such as sequence 700 in some embodiments, based on predetermined values of TD_{DIR} and TD_{PRESS}.

Double inversion recovery preparation pulses are applied at S810 during a first R-R interval and after the elapsing of TD_{DIR} from the first R peak of the first R-R interval. Flow cycles at S820 until a next R peak (i.e., a first R peak of a next R-R interval) is detected.

Navigator pulses are applied during the second R-R interval at S830. The temporal position of the navigator pulses is compared with a respiration cycle at S840 to determine if the temporal position is suitable. If not, flow returns to S810 at which double inversion recovery preparation pulses are applied during a next R-R interval.

If the temporal position of the navigator pulses is determined to be suitable at S840, flow proceeds to S850 through S880 as described above with respect to S430 to S460. In some embodiments, flow may return from S870 to S810 and repeat until it is determined that sufficient MR data has been acquired, at which point flow continues to S880.

Executable program code for dark-blood single-voxel spectroscopy with double inversion recovery according to the above description may be stored on a form of non-transitory computer-readable media. Computer-readable media includes volatile and nonvolatile, removable, and non-removable media implemented in any method or technology for storage of information such as program code, data structures, program modules or other data. Computer-readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access.

The foregoing diagrams represent logical architectures for describing processes according to some embodiments, and actual implementations may include more or different components arranged in other manners. Other topologies may be used in conjunction with other embodiments. Moreover, each component or device described herein may be implemented by any number of devices in communication via any number of other public and/or private networks. Two or more of such computing devices may be located remote from one another and may communicate with one another via any known manner of network(s) and/or a dedicated connection. Each component or device may comprise any number of hardware and/or software elements suitable to provide the functions described herein as well as any other functions. For example, any computing device used in an implementation of a system according to some embodiments may include a processor to execute program code such that the computing device operates as described herein.

Embodiments described herein are solely for the purpose of illustration. Those in the art will recognize other embodiments may be practiced with modifications and alterations to that described above.

What is claimed is:

1. A method for magnetic resonance (MR) spectroscopy, the method comprising:
applying double inversion recovery preparation pulses to a subject during a first R-R interval from a first R peak to a second R peak;
applying a plurality of localization pulses for spectroscopy to a portion of the subject during a second R-R interval from the second R peak to a third R peak, wherein application of the plurality of localization pulses is triggered by elapsing of a first delay time from the second R peak, the first delay time determined to substantially null a blood signal emitted from the portion of the subject;
acquiring MR data from the portion of the subject; and
determining a quantity of one or more metabolites in the region of interest based on the acquired MR data.

2. The method according to claim 1, wherein the double inversion recovery preparation pulses comprise a non-selective 180-degree radio frequency (RF) pulse followed by a selective 180-degree RF pulse.

3. The method according to claim 2, wherein the non-selective 180-degree RF pulse is configured to invert substantially all magnetization of the subject and the selective 180-degree RF pulse is configured to restore magnetization of a slice of the subject which includes the portion of the subject.

4. The method according to claim 1, wherein the plurality of localization pulses comprise point-resolved spectroscopy (PRESS) pulses.

5. The method according to claim 1, wherein the plurality of localization pulses comprise stimulated echo acquisition mode (STEAM) pulses.

6. The method according to claim 1, wherein one of the one or more metabolites are myocardial triglycerides.

7. The method according to claim 1, wherein one of the one or more metabolites is creatine.

8. The method according to claim 1, further comprising:
applying navigator pulses during the first interval and before application of the double inversion recovery preparation pulses.

9. The method according to claim 1, further comprising:
applying navigator pulses during the second interval and before application of the plurality of localization pulses.

10. The method according to claim 1, wherein a second delay time elapses between application of the double inversion recovery preparation pulses and application of the plurality of localization pulses, and the second delay time is determined as
$$[ ln ( 2 ) - ln ( 1 + {e}^{- \frac{2 R R}{T 1}} ) ] * {T}_{1} - {TD}_{P R E S S} ,$$
where RR is a heartbeat interval, T₁ is a longitudinal relaxation time of blood and TD_{PRESS} is the first delay time.

11. The method according to claim 10, wherein a third delay time (TD_{DIR}) elapses between the first R peak and application of the double inversion recovery preparation pulses, and the third delay time is determined as
$$T {D}_{DIR} = R R - \{ [ ln ( 2 ) - ln ( 1 + {e}^{- \frac{2 R R}{T 1}} ) ] * {T}_{1} - T {D}_{P R E S S} \} .$$

12. The method according to claim 10, wherein a second delay time (TD_{DIR}) elapses between the first R peak and application of the double inversion recovery preparation pulses, and the third delay time is determined as
$$T {D}_{DIR} = R R - \{ [ ln ( 2 ) - ln ( 1 + {e}^{- \frac{2 R R}{T 1}} ) ] * {T}_{1} - T {D}_{P R E S S} \} ,$$
where RR is a heartbeat interval, T₁ is a longitudinal relaxation time of blood and TD_{PRESS} is the first delay time.

13. A magnetic resonance imaging system comprising:
a magnet system configured to generate a polarizing magnetic field about at least a portion of a subject;
a plurality of gradient coils configured to apply at least one gradient field to the polarizing magnetic field;
a radio frequency (RF) system configured to apply an excitation field to the subject and to acquire magnetic resonance (MR) data from the subject; and
a processing unit to execute program code to cause the system to:
apply double inversion recovery preparation pulses to the subject during a first R-R interval from a first R peak to a second R peak;
apply a plurality of localization pulses to a portion of the subject during a second R-R interval from the second R peak to a third R peak, wherein application of the plurality of localization pulses is triggered by elapsing of a first delay time from the second R peak, the first delay time determined to substantially null a blood signal emitted from the portion of the subject;
acquire MR data from the portion of the subject; and
determine a quantity of one or more metabolites in the region of interest based on the acquired MR data.

14. The system according to claim 13, wherein the double inversion recovery preparation pulses comprise a non-selective 180-degree radio frequency (RF) pulse followed by a selective 180-degree RF pulse, and
wherein the non-selective 180-degree RF pulse is configured to invert substantially all magnetization of the subject and the selective 180-degree RF pulse is configured to restore magnetization of a slice of the subject which includes the portion of the subject.

15. The system according to claim 13, the processing unit to execute program code to cause the system to:
apply navigator pulses during the first interval and before application of the double inversion recovery preparation pulses.

16. The system according to claim 13, the processing unit to execute program code to cause the system to:
apply navigator pulses during the second interval and before application of the plurality of localization pulses.

17. The system according to claim 13, wherein a second delay time elapses between application of the double inversion recovery preparation pulses and application of the plurality of localization pulses, and the second delay time is determined as
$$[ ln ( 2 ) - ln ( 1 + {e}^{- \frac{2 R R}{T 1}} ) ] * {T}_{1} - T {D}_{P R E S S} ,$$
and
wherein a third delay time (TD_{DIR}) elapses between the first R peak and application of the double inversion recovery preparation pulses, and the third delay time is determined as
$$T {D}_{DIR} = R R - \{ [ ln ( 2 ) - ln ( 1 + {e}^{- \frac{2 R R}{T 1}} ) ] * {T}_{1} - T {D}_{P R E S S} \} ,$$
where RR is a heartbeat interval, T₁ is a longitudinal relaxation time of blood and TD_{PRESS} is the first delay time.

18. A non-transitory computer-readable medium storing program code executable by one or more processing units to cause a computing system to:
apply double inversion recovery preparation pulses to the subject during a first R-R interval from a first R peak to a second R peak;
apply a plurality of localization pulses to a portion of the subject during a second R-R interval from the second R peak to a third R peak, wherein application of the plurality of localization pulses is triggered by elapsing of a first delay time from the second R peak, the first delay time determined to substantially null a blood signal emitted from the portion of the subject;
acquire MR data from the portion of the subject; and
determine a quantity of one or more metabolites in the region of interest based on the acquired MR data.

19. The medium according to claim 18, wherein the double inversion recovery preparation pulses comprise a non-selective 180-degree radio frequency (RF) pulse followed by a selective 180-degree RF pulse, and
wherein the non-selective 180-degree RF pulse is configured to invert substantially all magnetization of the subject and the selective 180-degree RF pulse is configured to restore magnetization of a slice of the subject which includes the portion of the subject.

20. The medium according to claim 18, the program code executable by one or more processing units to cause a computing system to:
apply navigator pulses during the second interval and before application of the plurality of localization pulses.

21. The medium according to claim 18, wherein a second delay time elapses between application of the double inversion recovery preparation pulses and application of the plurality of localization pulses, and the second delay time is determined as
$$[ ln ( 2 ) - ln ( 1 + {e}^{- \frac{2 R R}{T 1}} ) ] * {T}_{1} - T {D}_{P R E S S} ,$$
and
wherein a third delay time (TD_{DIR}) elapses between the first R peak and application of the double inversion recovery preparation pulses, and the third delay time is determined as
$$T {D}_{DIR} = R R - \{ [ ln ( 2 ) - ln ( 1 + {e}^{- \frac{2 R R}{T 1}} ) ] * {T}_{1} - T {D}_{P R E S S} \} ,$$
where RR is a heartbeat interval, T₁ is a longitudinal relaxation time of blood and TD_{PRESS} is the first delay time.
